(19) United States
(12) Patent Application Publication
(10) Pub. No.: US 2023/0132147 A1
(43) Pub. Date: Apr. 27, 2023

(54) Foot-Actuated Device for Switching Flow Between Fluid Sources

(71) Applicant: Thoracent, Inc., Huntington, NY (US)

(72) Inventors: Felix Landaeta, Caguas, PR (US); Brian Lynch, Huntington, NY (US); Wissam Abouzgheib, Cherry Hill, NJ (US)

(21) Appl. No.: 18/069,531

(22) Filed: Dec. 21, 2022

Related U.S. Application Data
(63) Continuation-in-part of application No. 16/001,102, filed on Jun. 6, 2018.
(60) Provisional application No. 63/265,981, filed on Dec. 23, 2021.
(60) Provisional application No. 62/515,822, filed on Jun. 6, 2017.

(51) Int. Cl. A61M 16/20; F16K 31/62; A61M 39/28; A61M 16/10; A61M 1/00

(52) U.S. Cl. CPC A61M 16/208; F16K 31/62; A61M 39/28; A61M 16/1005; A61M 1/743; A61M 2205/078; A61M 2205/581

(57) ABSTRACT

A method and system for foot-actuated device to switch between a vacuum supply and an oxygen supply in a medical environment. The system enables a user to press down on the device to compress a first tube and open a second tube. When the user releases the device, the first tube is opened and the second tube is compressed.

### CROSS-REFERENCE

The present application relies on U.S. Patent Provisional Application No. 63/265,981, titled “Foot-Actuated Device for Switching Flow Between Fluid Sources” and filed on Dec. 23, 2021, for priority.

The present application is also a continuation-in-part application of U.S. patent application Ser. No. 16/001,102, titled “Apparatus, System and Method for Performing a Bronchoscopy” and filed on Jun. 6, 2018, which relies on U.S. Patent Provisional Application No. 62/515,822, titled “System and Method for Performing a Bronchoscopy” and filed on Jun. 6, 2017, for priority.

All of the above referenced application are incorporated herein by reference in their entirety.

### FIELD

Embodiments of the present specification relate to the field of medical devices. Specifically, the embodiments relate to a pedal device to enable switching between a vacuum supply and an oxygen supply.

### BACKGROUND

Medical devices such as bronchoscopes, laryngoscopes, and esophagoscopes are configured with channels for oxygen supply. At the same time, these devices require a channel for suction using vacuum. Conventional systems and methods require the operator of the system to press a button to apply vacuum.

For example, in US Patent Publication No. 2011/0264005, a bronchoscopy oxygenation system is used to permit the introduction of oxygen (or other gaseous substance) through a bronchoscopy device while also permitting various interventional procedures, for example, but not, limited to biopsy, lavage, and/or suction. In US Patent Publication No. 2015/0335228, bronchoscope adapters are described having a passageway, a mechanical ventilator access port, a bronchoscope access port configured to receive a bronchoscope into the passageway, and an exit port. The bronchoscope access port comprises a reversibly adjustable inner diameter component that provides locking of the bronchoscope at desired position. The adaptors find use in a variety of different applications.

In such systems, the operator must also be sure the supply of oxygen is switched off when they wish to provide suction. Conventional systems do not provide a simple means for switching between the supply of oxygen and applying suction to the various scopes. There is a need for efficient systems to alternatively operate the oxygen supply and the vacuum. What is needed are systems and methods that easily alternatively inject oxygen and apply vacuum, allowing the operator to re-oxygenate a patient through the same scope though which vacuum is applied.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a foot-actuated device for alternately switching fluid flow from a plurality of different fluid sources, comprising: at least one first port configured to connect to a first fluid source; at least one second port configured to connect to a second fluid source; at least one output port configured to connect to a medical device configured to provide fluid flow from at least one of the plurality of different sources to a patient; at least one first tube in fluid communication with the at least one first port; at least one second tube in fluid communication with the at least one second port; a tube connector in fluid communication with the at least one first tube, the at least one second tube, and the at least one output port; a first occluder configured to compress the at least one second tube; a pedal configured to be pressed and change a configuration of the foot-actuated device; and a second occluder configured to compress the at least one first tube; wherein, when the pedal is not pressed, the foot-actuated device is in a first configuration wherein the second occluder is not compressing the first tube and the first occluder is compressing the second tube, allowing a first fluid to flow from the first fluid source to the medical device and blocking a flow of a second fluid from the second fluid source to the medical device and, wherein, when the pedal is pressed, the foot-actuated device is in a second configuration wherein the second occluder is compressing the first tube and the first occluder is not compressing the second tube, allowing the flow of the second fluid from the second fluid source to the medical device and blocking the flow of the first fluid from the first fluid source to the medical device.

Optionally, the first fluid source comprises a vacuum source.

Optionally, the second fluid source comprises an oxygen source.

Optionally, the second occluder comprises a protrusion or column extending from a bottom surface of the pedal.

Optionally, the first occluder comprises a first portion configured to compress the at least one second tube and a second portion configured to contact a bottom surface of the pedal. Optionally, the foot-actuated device further comprises a first pivot point, wherein the first occluder is configured to pivot about the first pivot point to change the device from the first configuration to the second configuration. Optionally, the foot-actuated device further comprises a spring, wherein the first occluder is configured to automatically return the device to the first configuration by a spring-loaded force when the pedal is released. Optionally, the foot-actuated device further comprises a second pivot point positioned at a front portion of the pedal, configured to allow movement of a rear portion of the pedal relative to the front portion of the pedal.

Optionally, the foot-actuated device further comprises a check valve positioned between the at least one second port and the at least one second tube. Optionally, the check valve is configured to release the second fluid from the at least one second tube when a pressure in the at least one second tube reaches a predetermined threshold level.

Optionally, the foot-actuated device further comprises a notification mechanism configured to provide a first indication to an operator upon a change of the device from the first configuration to the second configuration and provide a second indication to the operator upon a change of the device from the second configuration to the first configuration. Optionally, the first indication and the second indication are different or are the same. Optionally, the first indication or the second indication comprises a clicking sound.

The present specification also discloses a method for operating a foot-actuated device for alternately switching fluid flow from a plurality of different fluid sources, wherein the foot actuated device comprises: at least one first port configured to connect to a first fluid source; at least one second port configured to connect to a second fluid source; at least one output port configured to connect to a medical device configured to provide fluid flow from at least one of the plurality of different sources to a patient; at least one first tube in fluid communication with the at least one first port; at least one second tube in fluid communication with the at least one second port; a tube connector in fluid communication with the at least one first tube, the at least one second tube, and the at least one output port; a first occluder configured to compress the at least one second tube; a pedal configured to be pressed and change a configuration of the foot-actuated device from a first configuration to a second configuration; and a second occluder configured to compress the at least one first tube, wherein, in the first configuration, the second occluder is not compressing the at least first tube and the first occluder is compressing the at least one second tube and, in the second configuration, the second occluder is compressing the at least one first tube and the first occluder is not compressing the at least one second tube, the method comprising: operating the medical device, when the clamp of the foot-actuated device is in the first configuration, to provide a first fluid from the first fluid source to the medical device; pressing the pedal to change the foot-actuated device from the first configuration to the second configuration, causing the second occluder to compress the at least one first tube and the first occluder to release from the at least one second tube, to provide a second fluid from the second fluid source to the medical device.

Optionally, the method further comprises releasing the pedal to change the foot-actuated device from the second configuration to the first configuration, causing the first occluder to compress the at least one second tube and release the second occluder from the at least one first tube, to provide the first fluid from the first fluid source to the medical device.

Optionally, the first fluid source comprises a vacuum source.

Optionally, the second fluid source comprises an oxygen source.

Optionally, the foot-actuated device further comprises a check valve positioned between the at least one second port and the at least one second tube and the method further comprises releasing the second fluid from the at least one second tube when a pressure in the at least one second tube reaches a predetermined threshold.

Optionally, the foot-actuated device further comprises a notification mechanism and the method further comprises the notification mechanism providing an operator a first indication upon a change of the foot-actuated device from the first configuration to the second configuration. Optionally, the method further comprises the notification mechanism providing a second indication to the operator upon a change of the foot-actuated device from the second configuration to the first configuration.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A illustrates an internal view of a foot-actuated device to switch between vacuum and oxygen supply, in accordance with some embodiments of the present specification;

FIG. 1B illustrates a top open view of the internal mechanism of a foot-actuated device to switch between vacuum and oxygen supply, in accordance with some embodiments of the present specification;

FIG. 2A illustrates a side elevation transparent view of a foot-actuated device to switch between vacuum and oxygen supply when a pedal is up (not pressed), in accordance with some embodiments of the present specification;

FIG. 2B illustrates a side elevation transparent view of a foot-actuated device to switch between vacuum and oxygen supply when the pedal is pressed, in accordance with some embodiments of the present specification;

FIG. 3 illustrates a perspective view of a one-way valve to be used with the foot-actuated device, positioned outside the pedal and configured to connect directly to a bronchoscope, in accordance with some embodiments of the present specification; and

FIG. 4 is a flowchart listing the steps in a method of alternately providing vacuum suction and oxygen to a medical device using a foot-pedal device, in accordance with some embodiments of the present specification.

### DETAILED DESCRIPTION

The present specification is directed toward a foot-actuated device to switch between a vacuum supply and an oxygen supply. The foot-actuated device is configured to be used with endoscopes to alternately input oxygen into, and provide suction from, the same scope. The device is actuated by pressing a foot on a pedal. Actuating the device by pressing down on a foot pedal compresses a first tube, for example, a vacuum supply tube, and opens up a second tube, for example, an oxygen supply tube, shutting off vacuum suction from the endoscope and providing oxygen to the endoscope and to the patient. When the foot is removed and the foot pedal is released, the vacuum supply tube is opened and the oxygen supply tube is compressed, shutting off the flow of oxygen and providing suction to the scope. In other words, in some embodiments, the default or un-actuated mode of the device provides vacuum suction and oxygen delivery is blocked. In embodiments, the first tube leads to a vacuum source and the second tube leads to an oxygen source. In other, alternative embodiments, the first tube leads to an oxygen source and the second tube leads to a vacuum source such that, when the food pedal is pressed, vacuum is supplied and oxygen delivery is blocked. A check valve is configured at an intersection of a spring and a tubing connector within the foot-actuated device. The check valve is configured to actuate manually upon pressing of the pedal. In embodiments, when the pedal is released, a column releases a line holding vacuum and closes a line holding oxygen. When the pedal is pressed, it goes down and closes the vacuum line and opens the oxygen line. The two lines merge into one line that goes to a system, such as a medical system. Embodiments of the medical system include and are not limited to bronchoscopes, laryngoscopes, and esophagoscopes. In some embodiments, a notification mechanism is included in the foot-actuated device and is configured to indicate to the operator that the switch between vacuum and oxygen has been made. In some embodiments, the notification mechanism comprises a clicking mechanism configured to generate an audio indication in the form of a clicking sound to indicate to a user that a change has been triggered. In some embodiments, occluders are configured in the device of the present specification. When oxygen line is occluded, a check valve releases air from the flow of the oxygen to atmosphere.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems and methods are now described.

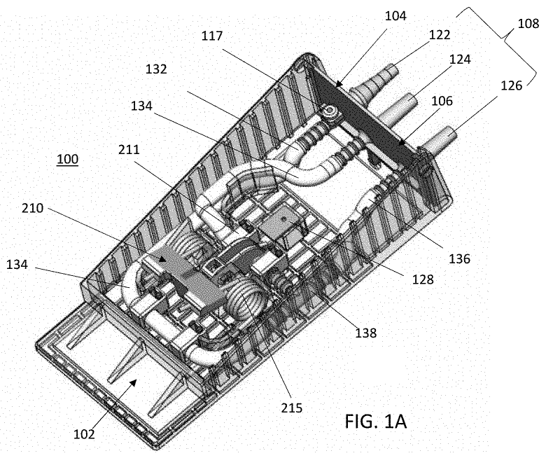

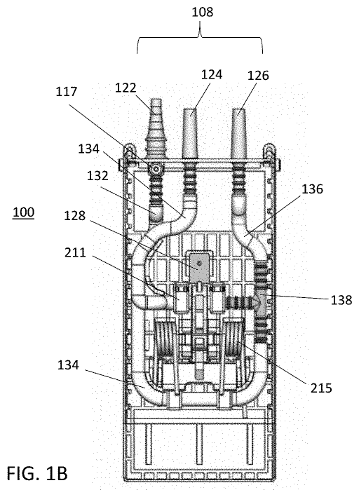

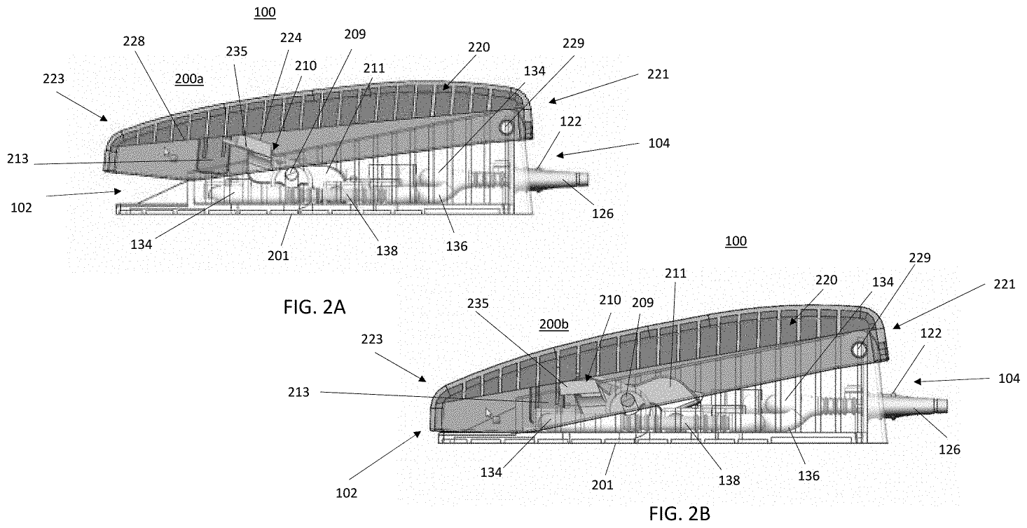

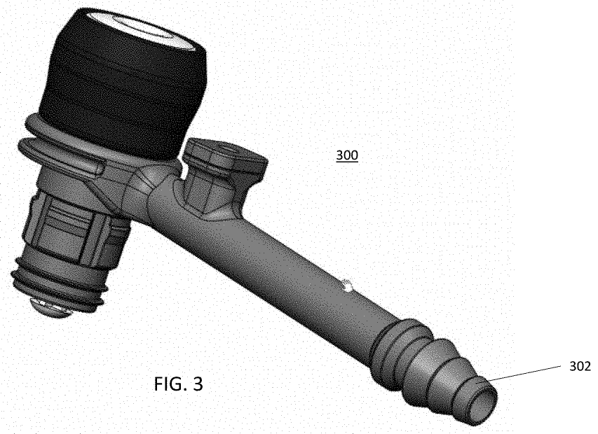

FIG. 1A illustrates an internal view of a foot-actuated device 100 to switch between vacuum and oxygen supply, in accordance with some embodiments of the present specification. FIG. 1B illustrates a top open view of the internal mechanism of device 100 in accordance with some embodiments of the present specification. Referring to FIGS. 1A and 1B, the top surface or pedal has been removed to assist with visualization of the components within. FIG. 2A illustrates a side elevation transparent view 200a of device 100 when a pedal 220 is up (not pressed) and the device 100 is in a first or default state or mode, in accordance with some embodiments of the present specification. In the position illustrated in FIG. 2A, an oxygen tube is compressed or closed and a vacuum tube is not compressed or open. FIG. 2B illustrates a side elevation transparent view 200b of device 100 when pedal 220 is pressed and the device 100 is in a second or actuated state or mode, in accordance with some embodiments of the present specification. In the position illustrated in FIG. 2B, an oxygen tube is not compressed or open, a check valve 117 is closed, and a vacuum tube is compressed or closed. FIG. 3 illustrates a perspective view of a valve 300 to be used with the foot-actuated device, positioned outside the pedal and configured to connect directly to a device, such as an aspirator or bronchoscope, in accordance with some embodiments of the present specification. In embodiments, a first end of a suction tube is configured to connect to a first connector 302 of the valve 300 and a second end of the suction tube is configured to connect to output port 126 of device 100 shown in FIGS. 1A-2B. In embodiments, the valve 300 is an on-off valve.

Referring to FIGS. 1A, 1B, 2A, 2B, and 3 simultaneously, device 100 is configured in the form of a three-dimensional ramp. A bottom surface 201 of device 100 can be placed in a horizontal plane and is rectangular in form. A top surface, comprising a pedal 220, opposite to the bottom surface 100 is also rectangular and configured at an angle to the bottom surface, creating a ramp or an inclination over which a user can place a foot. All four sides, between the top surface and the bottom surface are covered. In some embodiments, the surfaces are composed of a polymeric material, such as polypropylene or polycarbonate. In some embodiments, the foot-pedal device 100 has a length in a range of 140 mm to 200 mm, a width in a range of 70 mm to 94 mm, and a height in a range of 45 mm to 55 mm, with a pedal angle in a range of 10 to 30 degrees when closed/compressed. In one embodiment, the foot-pedal device 100 has a length of 170 mm, a width of 82 mm, and a height of 50 mm, with a pedal angle of 20 degrees when closed/compressed. During operation, device 100 is preferably positioned such that when the user places the foot on the external side of the top surface or pedal 220, the ankle rests near a lower edge 102 of the top surface and the toes rest near a higher edge 104 of the inclined top surface. Higher edge 104 is the top edge of the planar surface 106 that connects the top and bottom surfaces of device 100 on a distal or front side. Ports 108 are fixedly positioned through planar surface 106, two of which are input port which provide oxygen and vacuum suction and the third of which is an output port to the patient. A first port 124 provides input for vacuum to a medical device, such as an endoscope, connected to device 100. A second port 122 provides an input for oxygen source to the medical device connected to device 100, and a third port (output port) 126 provides an output for either vacuum suction or oxygen, depending on the operational state or mode of the device 100, to the medical device attached to the foot-actuated device 100. Each port 108 extends into the device 100, wherein the first port 124 connects to and is in fluid communication with a corresponding vacuum tube 134, the second port 122 connects to and is in fluid communication with a corresponding oxygen tube 132, and the third port (output port) 126 connects to and is in fluid communication with a corresponding output tube 136. Vacuum tube 134 and oxygen tube 132 connect to, and are in fluid communication with one another, and with output tube 136, via tube connector 138. In some embodiments, tube connector 138 is a T-shaped connector. In embodiments, at least one check valve 117 is positioned between oxygen tube 132 and the second port 122 and is configured to release oxygen from oxygen tube 132 into the atmosphere when a pressure in the oxygen tube 132, as measured by the check valve 117, reaches a predetermined pressure threshold.

During operation, when rear portion 223 of pedal 220 of device 100 is up (not compressed), such that the device 100 is in a first, default state or mode of operation (depicted in FIG. 2A), vacuum occluder 213, extending downward from an inner surface of pedal 220, is not compressing vacuum tube 134 such that vacuum tube 134 is open, allowing vacuum to be applied, from vacuum port 124 though vacuum tube 134 and tube connector 138, to a medical device connected at output port 126. When the vacuum tube 134 is open, the device 100 is in a first configuration or first, default state and an oxygen occluder 210 is compressing the oxygen tube 132, allowing vacuum flow and blocking oxygen flow. In some embodiments, the vacuum occluder 213 comprises a column. A first portion 211 of oxygen occluder, or clamp 210 is compressing oxygen tube 132, occluding oxygen tube 132 and preventing the flow of oxygen to the medical device. Oxygen occluder 210 is configured to pivot about a first, clamp pivot point 209. Actuating pedal 220 by pressing or stepping on it, such that the rear portion 223 of the pedal 220 of device 100 is down, causes oxygen occluder 210 to pivot about the first, clamp pivot point 209 such that the first portion 211 of the oxygen occluder 210 lifts up, releasing the compression from oxygen tube 132, allowing oxygen to flow from oxygen port 122 through oxygen tube 132 and tube connector 138 to a medical device connected at output port 126. Simultaneously, the vacuum occluder 213 compresses vacuum tube 134, effectively shutting off the vacuum during oxygen delivery by occluding vacuum tube 134 and preventing vacuum suction from vacuum port 124. A second, pedal pivot point 229, positioned proximal a front portion 221 of the pedal 220, allows movement of the rear portion 223 of the pedal 220 relative to the front portion 221. When the oxygen tube 132 is open, the vacuum occluder 213 is compressing the vacuum tube 134 and the device 100 is in a second configuration or actuated state, allowing oxygen flow and blocking vacuum flow.

Oxygen occluder 210 is spring-loaded via spring 215. When pedal 220 is released, oxygen occluder 210 is configured to pivot about the first, clamp pivot point 209, as a result of the spring-loaded force from spring 215, such that the vacuum occluder 213 of the pedal 220 moves up and releases compression from the vacuum tube 134. A top surface 224 of a second portion 235 of the oxygen occluder 210 is in physical contact with a bottom (inner) surface 228 of the pedal 220 and upward movement of the second portion 235 of the oxygen occluder 210 causes the rear portion 223 of the pedal 220 to move upward, returning the operational mode or state of the device 100 to the first, default state or mode. The first portion 211 of the oxygen occluder 210 moves down, as a result of the spring-loaded force of the spring 215, to compress the oxygen tube 132 and occlude oxygen flow. Depending on the operational mode or state of the device 100, tube connector 138 receives, and delivers to output port 136, either vacuum suction from the vacuum tube 134 (when the device 100 is in the first, default state or mode) or oxygen via oxygen tube 132 (when the device 100 is in the second, actuated state or mode). Passage of vacuum suction or oxygen is blocked from entering the tube connector 138 when the corresponding tube is compressed. When the device 100 is in the first, default mode or state, the oxygen tube 132 is compressed by the first portion 211 of oxygen occluder 210 (while the vacuum tube 134 is open) and oxygen is blocked from flowing through oxygen tube 132 and into tube connector 138. When the device 100 is in the second, actuated mode, the vacuum tube 134 is compressed by the vacuum occluder 213 of the pedal 220 (while the oxygen tube 132 is open) and vacuum suction is blocked from flowing through the vacuum tube 134 and into tube connector 138. The medical device attached via output port 126 may be a bronchoscope, laryngoscope, esophagoscope, or any other endoscope or system that requires switching supply between two different sources such as vacuum and oxygen.

Device 100 is configured to connect to a one-way valve 300. The one-way valve 300 facilitates connection of the foot-actuated device of the present specification directly to a medical device, such as a bronchoscope.

In some embodiments, a notification mechanism 128 is included in the foot-actuated device 100 and is configured to indicate to the operator that the switch between vacuum and oxygen has been made. In some embodiments, the notification mechanism 128 is configured to provide a first indication to the operator when the foot-actuated device changes from vacuum to oxygen flow and a second indication to the operator when the foot-actuated device changes back to vacuum flow from oxygen flow. In some embodiments, the first and second indications are different. In other embodiments, the first and second indications are the same. In some embodiments, the notification mechanism 128 comprises a clicking mechanism configured to generate an audio indication in the form of a clicking sound to indicate to a user that a change has been triggered. In some embodiments, the clicking mechanism makes a first sound when the pedal 220 is pressed, indicating a switch has been made from vacuum flow to oxygen flow. In some embodiments, the clicking mechanism makes a second sound when the pedal 220 is released, indicating a switch has been made from oxygen flow to vacuum flow. In some embodiments, the first sound is different than the second sound. In some embodiments, the first sound is the same as the second sound.

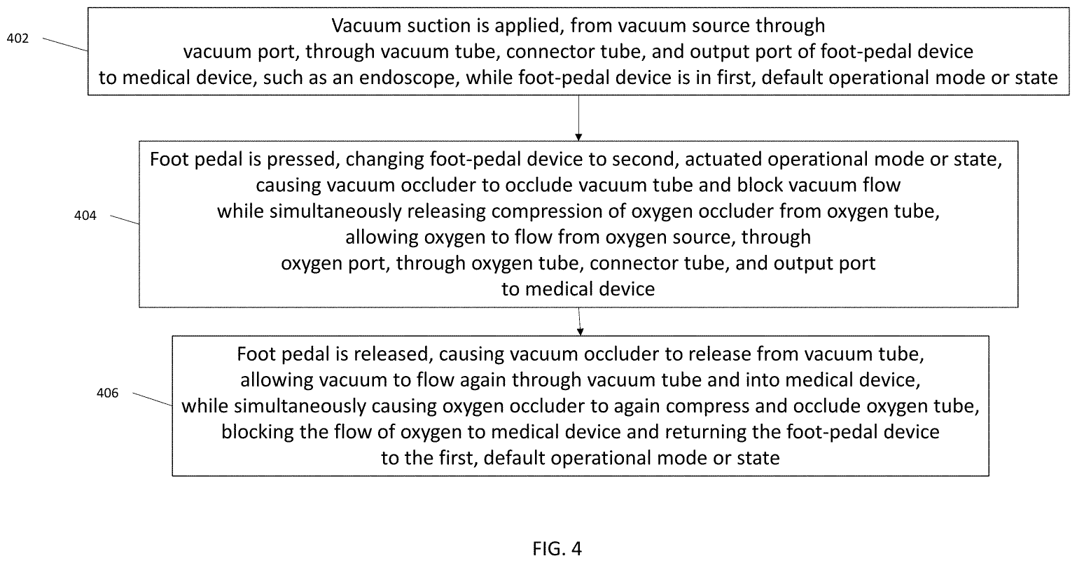

FIG. 4 is a flowchart listing the steps in a method of alternately providing vacuum suction and oxygen to a medical device using a foot-pedal device, in accordance with some embodiments of the present specification. The foot-pedal device is similar to the foot-pedal device described with reference to FIGS. 1A-3. At step 402, vacuum suction is applied, from vacuum source through a vacuum port, through a vacuum tube, a connector tube, and an output port of the foot-pedal device to a medical device, such as an endoscope, while the foot-pedal device is in a first, default operational mode or state. The foot pedal is pressed, at step 404, changing the foot-pedal device to a second, actuated operational mode or state, causing a vacuum occluder to occlude a vacuum tube and block vacuum flow while simultaneously releasing compression of an oxygen occluder from an oxygen tube, allowing oxygen to flow from an oxygen source, through an oxygen port, through the oxygen tube, the connector tube, and the output port to the medical device. At step 406, the foot pedal is released, causing the vacuum occluder to release from the vacuum tube, allowing vacuum to flow again through the vacuum tube and into the medical device, while simultaneously causing the oxygen occluder to again compress and occlude the oxygen tube, blocking the flow of oxygen to the medical device and returning the foot-pedal device to the first, default operational mode or state.

Although the present specification has been described with particular focus on a pedal device mechanism for use with a medical device, the present specification is also designed to be used with other devices where pedal mechanism may be used to alternate between two different sources.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A foot-actuated device for alternately switching fluid flow from a plurality of different fluid sources, comprising:
at least one first port configured to connect to a first fluid source;
at least one second port configured to connect to a second fluid source;
at least one output port configured to connect to a medical device configured to provide fluid flow from at least one of the plurality of different sources to a patient;
at least one first tube in fluid communication with the at least one first port;
at least one second tube in fluid communication with the at least one second port;
a tube connector in fluid communication with the at least one first tube, the at least one second tube, and the at least one output port;
a first occluder configured to compress the at least one second tube;
a pedal configured to be pressed and change a configuration of the foot-actuated device; and
a second occluder configured to compress the at least one first tube;
wherein, when the pedal is not pressed, the foot-actuated device is in a first configuration wherein the second occluder is not compressing the first tube and the first occluder is compressing the second tube, allowing a first fluid to flow from the first fluid source to the medical device and blocking a flow of a second fluid from the second fluid source to the medical device and, wherein, when the pedal is pressed, the foot-actuated device is in a second configuration wherein the second occluder is compressing the first tube and the first occluder is not compressing the second tube, allowing the flow of the second fluid from the second fluid source to the medical device and blocking the flow of the first fluid from the first fluid source to the medical device.

2. The foot-actuated device of claim 1, wherein the first fluid source comprises a vacuum source.

3. The foot-actuated device of claim 1, wherein the second fluid source comprises an oxygen source.

4. The foot-actuated device of claim 1, wherein the second occluder comprises a protrusion or column extending from a bottom surface of the pedal.

5. The foot-actuated device of claim 1, wherein the first occluder comprises a first portion configured to compress the at least one second tube and a second portion configured to contact a bottom surface of the pedal.

6. The foot-actuated device of claim 5, further comprising a first pivot point, wherein the first occluder is configured to pivot about the first pivot point to change the device from the first configuration to the second configuration.

7. The foot-actuated device of claim 6, further comprising a spring, wherein the first occluder is configured to automatically return the device to the first configuration by a spring-loaded force when the pedal is released.

8. The foot-actuated device of claim 7, further comprising a second pivot point positioned at a front portion of the pedal, configured to allow movement of a rear portion of the pedal relative to the front portion of the pedal.

9. The foot-actuated device of claim 1, further comprising a check valve positioned between the at least one second port and the at least one second tube.

10. The foot-actuated device of claim 9, wherein the check valve is configured to release the second fluid from the at least one second tube when a pressure in the at least one second tube reaches a predetermined threshold level.

11. The foot-actuated device of claim 1, further comprising a notification mechanism configured to provide a first indication to an operator upon a change of the device from the first configuration to the second configuration and provide a second indication to the operator upon a change of the device from the second configuration to the first configuration.

12. The foot-actuated device of claim 11, wherein the first indication and the second indication are different.

13. The foot-actuated device of claim 11, wherein the first indication and the second indication are the same.

14. The foot-actuated device of claim 11, wherein the first indication or the second indication comprises a clicking sound.

15. A method for operating a foot-actuated device for alternately switching fluid flow from a plurality of different fluid sources, wherein the foot actuated device comprises: at least one first port configured to connect to a first fluid source; at least one second port configured to connect to a second fluid source; at least one output port configured to connect to a medical device configured to provide fluid flow from at least one of the plurality of different sources to a patient; at least one first tube in fluid communication with the at least one first port; at least one second tube in fluid communication with the at least one second port; a tube connector in fluid communication with the at least one first tube, the at least one second tube, and the at least one output port; a first occluder configured to compress the at least one second tube; a pedal configured to be pressed and change a configuration of the foot-actuated device from a first configuration to a second configuration; and a second occluder configured to compress the at least one first tube, wherein, in the first configuration, the second occluder is not compressing the at least first tube and the first occluder is compressing the at least one second tube and, in the second configuration, the second occluder is compressing the at least one first tube and the first occluder is not compressing the at least one second tube, the method comprising:
operating the medical device, when the clamp of the foot-actuated device is in the first configuration, to provide a first fluid from the first fluid source to the medical device;
pressing the pedal to change the foot-actuated device from the first configuration to the second configuration, causing the second occluder to compress the at least one first tube and the first occluder to release from the at least one second tube, to provide a second fluid from the second fluid source to the medical device.

16. The method of claim 15, further comprising releasing the pedal to change the foot-actuated device from the second configuration to the first configuration, causing the first occluder to compress the at least one second tube and release the second occluder from the at least one first tube, to provide the first fluid from the first fluid source to the medical device.

17. The method of claim 15, wherein the first fluid source comprises a vacuum source.

18. The method of claim 15, wherein the second fluid source comprises an oxygen source.

19. The method of claim 15, wherein the foot-actuated device further comprises a check valve positioned between the at least one second port and the at least one second tube, the method further comprising releasing the second fluid from the at least one second tube when a pressure in the at least one second tube reaches a predetermined threshold.

20. The method of claim 15, wherein the foot-actuated device further comprises a notification mechanism, the method further comprising the notification mechanism providing an operator a first indication upon a change of the foot-actuated device from the first configuration to the second configuration.

21. The method of claim 20, further comprising the notification mechanism providing a second indication to the operator upon a change of the foot-actuated device from the second configuration to the first configuration.
